(19) United States
(12) Patent Application Publication
(10) Pub. No.: US 2025/0169814 A1
(43) Pub. Date: May 29, 2025

(54) AUTOMATED SKIN CLOSURE SYSTEM

(71) Applicant: Opus KSD Inc., Pembroke, MA (US)

(72) Inventors: Charles H. Rogers, Halifax, MA (US); Eduard Ulise Milea, Irvine, CA (US)

(21) Appl. No.: 18/964,231

(22) Filed: Nov. 29, 2024

Related U.S. Application Data
(60) Provisional application No. 63/603,769, filed on Nov. 29, 2023.

(51) Int. Cl. A61B 17/068; A61B 17/00; A61B 17/08

(52) U.S. Cl. CPC A61B 17/068; A61B 2017/00004; A61B 2017/081

(57) ABSTRACT

The present invention is directed to a surgical system, including a device for deploying bioabsorbable fasteners into tissue to secure portions of tissue surrounding an incision or wound in the skin of a patient to effectively close the incision or wound.

### CROSS-REFERENCE TO RELATED APPLICATIONS

This application claims priority to, and the benefit of, U.S. Provisional Application No. 63/603,769, filed Nov. 29, 2023, the content of which is incorporated by reference herein in its entirety.

### FIELD OF THE INVENTION

The present invention generally relates to medical devices, and, more particularly, to a surgical system, including a device for deploying bioabsorbable fasteners into tissue to secure portions of tissue surrounding an incision or wound in the skin of a patient to effectively close the incision or wound.

### BACKGROUND

When manually applying sutures to close incisions or wounds in tissue, a suture is stitched through the tissue and tied to hold edges of the wound or incision together to promote healing. With the development of synthetic absorbable materials, it has become common to use a bioabsorbable suture material which is inserted beneath the skin surface and is resorbed by the body once healing is complete. There are several different techniques involving the path of the needle, where to place knots, etc., in order to engage the strong dermal layer and provide a secure closure. The dermal layer, which is typically less than two (2) millimeters thick, is the strongest of the three layers that make up human skin tissue. The surgeon “feels” the resistance of the needle as it penetrates the dense dermal tissue and skillfully guides it to enter and exit at a depth and with a sufficient size “bite” judged to be appropriate for the tissue and the stress that the wound may experience during healing.

While manual suturing remains a popular means for closing incisions and wounds, such a procedure can be time consuming, requires a certain level of skill, and poses the risk of needle accidents (e.g., inadvertent needle pricks and the like). Accordingly, many practitioners now use surgical staplers which deliver staples into tissue to hold an incision closed for healing. These surgical staplers deliver metal staples which have sharp tips enabling them to pierce the tissue. When deployed, the stapler device bends the metal staple into a closed shape to hold the tissue edges together. One of the disadvantages of metal staples is that a portion of the metal must remain exposed through the skin surface in order to allow a medical professional to remove the staple once biological healing is complete. This exposed portion is unsightly, and the puncture points, where the staple enters the skin, have a risk of infection. Moreover, the delayed healing of these percutaneous points fills in with scar tissue which often results in a “railroad track” appearance with the linear incision line laterally flanked by pairs of small points where the staples pierced the skin.

To address the disadvantages of metal staples, various inventors have proposed fasteners made of bioabsorbable materials which can be placed below the surface of the skin. One such device is described in U.S. Pat. No. 8,506,591 issued to Danielson et al. (“Danielson”) titled “Tissue Fasteners and Related Insertion Devices, Mechanisms, and Methods”. Danielson teaches a device that requires two people to operate: one person using forceps in each hand to evert the two sides of the incision, and a second person to operate the stapler. More recently U.S. Pat. No. 11,826,049 issued to Rogers et al. (“Rogers”) titled “Devices for Deploying Tissue Fasteners”, describes devices operable by a single user which incorporate into the device itself capabilities to perform some of the tasks normally done by a second operator.

All of the existing methods and devices that are used to close incisions require surgically trained operators with significant skill, training and experience. Closing the skin, however, is the last task in what sometimes is a long procedure and the specialist(s) who performed that procedure may be tired or needed in another case. Leaving the skin closure to less skilled personnel has the risk of complications, surgical site infections, or an unattractive scar. Independent of who performs the skin closure, it can be tedious and time consuming.

### SUMMARY

The present invention improves upon and extends the capabilities of the devices described by Danielson and Rogers. In particular, the present invention addresses the limitations of current methods and devices by providing an automated skin closure system configured to operate with minimal input from skilled operators, and without requiring a surgeon. The system is able to quickly deploy bioabsorbable fasteners to obtain a secure skin closure of a wound or incision that will heal without complications and yield a minimal scar. The present invention makes it possible to automate skin closure for a large number of surgical procedures.

Closing a wound or a surgical incision in a manner that will heal without complication and yield a minimal scar has multiple requirements. The present invention achieves the desired results by implementing some or all of the eight (8) inventive elements described herein.
1. As an initial matter, the system utilizes a bioabsorbable fastener comprising two legs and configured to penetrate opposing sides of an incision or wound. As will be described in greater detail herein, the system is configured to deploy bioabsorbable fasteners of the type set forth and described in Danielson. However, it should be noted that other embodiments of bioabsorbable fasteners are contemplated to be used with and deployed by the disclosed system. To meet the requirements of the present invention, a given bioabsorbable fastener has two legs configured to penetrate tissue, but further configured to resist reverse movement or removal. Such anti-reversing capability includes, but is not limited, to: barbs oriented to resist backward movement; toggles which create a larger profile after penetration so that they cannot return through the same path; or legs which latch together after separately penetrating opposing sides of the incision or wound. Traditional staples do not meet these requirements, as they hold the tissue by maintaining a closed “horseshoe” shape and require strong bending strength. A critical distinction of the bioabsorbable fastener of the present invention is that it is designed to bend easily and holds opposing sides of the wound or incision together by the tensile strength of a connecting element between the two legs.

The bioabsorbable fastener is deployed by a fastener delivery mechanism containing a plurality of fasteners within a magazine. Fasteners are deployed in one-by-one fashion by mechanical elements, including a plunger needle assembly and an insertion device that travels in guide-tracks and passes through the distal end of the fastener delivery mechanism.
2. According to the present invention, tissue at opposing sides of an incision is positioned adjacent an exit portion of the fastener delivery mechanism such that when the bioabsorbable fastener passes through the exit, the point where each fastener leg enters the tissue is set back from the cut edge. The meaning of “set-back” comes from the manual procedure called the Set-back Buried Dermal Suture technique, an interrupted vertical stich first published by Jonathan Kantor in 2010 (J. American Academy Dermatology, 2010 February, pg 352-353, “Letters to the Editor”, Kantor, Jonathan). Wang et al (J Am Acad Dermatol, 2015 April, 72(4), 674-680, “Set-back versus buried vertical mattress suturing: Results of a randomized blinded trial”, Wang A S, Kleinerman, R, Armstrong, A W, Fitzmaurice, S, Pascucci A, Awasthi S, Ratnarathorn M, Sivamani R, King T H, Eisen D B) showed the Set-Back Technique has superior wound eversion, provided better cosmetic outcomes, and had fewer spitting sutures compared to traditional suture technique. The present invention improves upon the set-back technique by controlling the set-back distance according to the geometry of the fastener. Controlling the set-back distance provides uniformity to the eversion of the opposed edges and distributes the forces that might rupture the incision. The amount of “eversion” refers to the tenting-up of the tissue edges which helps to keep the edges in close contact even when the tissue is subjected to movement or forces pulling on the tissue.
3. The system is able to position the tissue with reference to the outer skin surface so that the path of the fastener legs is a controlled distance from the outer skin surface, or epidermis. The epidermis is the thinnest of the three layers of human skin, typically less than 1 mm. The dermal layer is immediately below the epidermis. The dermal layer is the strongest tissue and varies in thickness between 1 and 4 mm. The subcutaneous layer is the innermost layer of the skin and consists of a network of fat and collagen cells and does not have the strength to hold the incision closed. In one embodiment the fastener path is controlled to be a specified distance from the outer skin surface which reliably locates it within the dermal layer independent of the overall thickness of the skin tissue and its various layers. We have determined that “D”, the distance between the outer-most surface of the fastener legs and outer skin surface, should not exceed 0.27 mm. and should typically be 0.17 mm when the tissue is in position for fastener insertion.
4. In one embodiment of the present invention, exceptional consideration is given to maintaining the critical distance between the outer skin surface and the fastener during the insertion of the fastener. This means that the frontal surface of the foot where it contacts the outer skin surface must not flex, compress or move to any significant extent when the fastener penetrates the tissue. In one embodiment, the foot is part of a tissue holder which is moved into position by a mechanism to position tissue for fastener insertion. We have determined that this mechanism must have a stiffness to resist backward movement. “Stiffness” as used herein, is the force gradient, the derivative of force with respect to distance, df/dx. We have determined that the stiffness of the mechanism measured by applying force (df) and measuring movement (dx) at the frontal surface of the tissue holder/foot where it contacts the outer skin surface, must be more than 20 Lbf/mm when the foot is in position for deploying the fastener. The present invention achieves the required mechanical stiffness by driving the tissue holder/foot against “stops” that block further motion once the tissue holder/foot has reached the desired location. In one embodiment, the force pushing the tissue holder/foot against the “stops” is between 8 and 15 Lbf. In an alternate embodiment the force that the tissue holder applies to the “stops” is applied for a short time-period after contacting the “stops”, (so called “over-driving”), to compress the pushing element(s) a predetermined distance to ensure that the mechanism resists backward movement of the tissue holder/foot.
5. In order to simultaneously engage the thin dermal layer on both sides of the incision, it is important to have the same spacing between the fastener and the tissue on both sides. Small differences between components on the left versus right side of the device—in terms of dimensions or flexibility or friction—can result in differences in the forces that press the tissue into position. If the differences are great enough, the fastener may not engage the dermis on one side putting the incision at risk of rupturing. One embodiment of the present invention provides a means to reduce this risk or completely neutralize it by providing a centering mechanism to balance the forces and ensure equal spacing between the fastener and the foot on each side of the incision. This is accomplished by rotatably attaching the proximal end of the fastener delivery mechanism and allowing the distal end to move laterally by a small amount in response to forces exerted by the tissue and tissue holders. As an example, consider that tissue holders on one side of the device exert 2× the force as compared to the other. When the stronger tissue holder contacts the fastener delivery mechanism, the fastener delivery mechanism moves, thereby reducing the force; movement away from one side is necessarily toward the other side, which increases the effective force on that side. Thus, any initial imbalance in the force being applied by one side versus the other is neutralized by the movement of the delivery mechanism until the forces are balanced. In this way the fastener delivery mechanism and the tracks guiding the insertion device are centered between the tissue holders/feet.
6. The insertion device carrying the bioabsorbable fastener moves in guide-tracks that bring the fastener through an “exit” in the distal end of the fastener delivery mechanism. To move freely, there must be clearance between the fixed and moving parts, typically +/−0.1 mm of space. To ensure that this space is available in every device, one must also allow for tolerances in the dimensions of the parts, which for injection molding is typically +/−0.06 mm. Unfortunately, the stack-up of these tolerances can be greater than the allowable range of positions that the fastener must hit. In one embodiment of the present invention the uncertainty of the fastener path is reduced by actively altering the dimensions of the exit. During the initial movement of the insertion device, the exit is opened approximately 0.2 mm to provide freedom of movement. The exit is then closed to an interference fit with the fastener for precise guidance of the fastener as it passes through the exit and enters the tissue. Once the fastener is released into the tissue, the exit is again opened to allow for free movement of the mechanism to its initial position.
7. When a surgeon inserts a needle with traditional suture material, the surgeon adjusts the force being applied as needed to pierce the tissue. If the tissue is stretchy, the needle is pushed as far as needed to penetrate the dermis. The surgeon may not even realize how much adjustment is being applied from one patient to the next and one body area to another. To automate skin closure, the present invention must adapt the forces and/or depth of insertion of the fastener in response to differences in tissue properties. In one embodiment, electrical or other motive forces having sensors that provide feedback on position and force are configured to move the tissue holders and the insertion device. We have measured the force versus distance curves for inserting needles compared to similar measurements with a barbed fastener. The data for the barbed fasteners have a distinct profile which can be analyzed in real time and used to halt further insertion once the barb of the fastener is fully engaged.
8. In one embodiment of the present invention, a suture insertion device is a replaceable component of an automated system further comprising one or more graspers, a vision system controlled by software enabled by artificial intelligence, and a programmable controller operably coupled to the one or more graspers, the vision system, and the suture insertion device. As described in greater detail herein, the controller is configured to effect electro-mechanical control of the one or more graspers and one or more components of the suture insertion device in response to input received via one or more sensors, including input from the vision system, to provide autonomous, or semi-autonomous, delivery of bioabsorbable fasteners to secure opposing sides of an incision to effectively close the incision and promote healing.

As previously described, the inventive elements of the suture insertion device provide accurate and reliable placement of the fasteners while only requiring simple positioning of the tissue. In particular, such positioning preferably is accomplished by three (3) independent graspers, one at the apex of the incision in front of the suture insertion device and two at the back. The system is positioned over the patient so that the camera field of view and the mechanical elements can identify and reach the incision(s) to be closed. For each incision, the front grasper is used to lift the tissue at the apex while the other two graspers lift and open the two edges of the incision behind the suture insertion device. While the graspers lift and open the incision, an “introducer” feature at the distal end of the fastener delivery mechanism is lowered to a predetermined depth and moved to the apex. The motors controlling the tissue holders move to partially engage the tissue. If the vision system confirms that the position is correct, the tissue holders are driven to their final positions, and the motor driving the insertion device deploys the bioabsorbable fastener using feedback to control the depth. The insertion motor is then reversed to partially retract the needles for protection, the motors controlling the tissue holders reverse and release the tissue, and finally all actuators return to their original positions. If another fastener is to be deployed, the suture insertion device is directed to move longitudinally along the incision, away from the apex, a preset distance between 7 and 10 mm and to re-engage the tissue with the tissue holders. While the tissue holders secure the tissue, all three graspers move a distance corresponding to the distance that the suture insertion device moved and re-grasp the tissue edges. These steps are repeated until the incision is closed. As additional fasteners are inserted, the graspers at the back reach the end of the incision and are directed by the controller to disengage and move away. When the incision is fully closed, the entire device is lifted and removed from the sterile field.

In one aspect, the present invention is directed to an automated system for closing one or more incisions or wounds of a patient. The system includes a movable platform and a suture insertion device releasably coupled to a portion of the movable platform. The suture insertion device is configured to deploy bioabsorbable fasteners to close an incision or wound in tissue of a patient. The suture insertion device may generally be a single use device. Accordingly, the movable platform is configured to receive and releasably retain interchangeable suture insertion devices.

The suture insertion device includes a body comprising a pair of tissue holders positioned adjacent to a distal end of the body and operably coupled to first and second actuators associated with the movable platform, each of the first and second actuators being configured to move the respective tissue holder to effectively pinch and fold one edge of the incision or wound in preparation for deployment of a fastener. The suture insertion device further includes a fastener delivery mechanism operably coupled to an insertion actuator associated with the movable platform and configured to deploy one or more of a plurality of bioabsorbable fasteners. The fastener delivery mechanism includes an insertion device comprising one or more insertion needles, said insertion device being guided within tracks and configured to move in response to movement of the insertion actuator to cause the one or more insertion needles to releasably engage and deploy each one of the plurality of fasteners in a one-by-one fashion in response to repeated movements of the insertion actuator. The fastener delivery mechanism also includes an introducer positioned at, and extending from, a distal end of the fastener delivery mechanism, the introducer configured to directly contact opposing edges of the wound or incision and having an opening through which a fastener exits the distal end of the introducer during deployment. The fastener delivery mechanism further includes a pair of stopping surfaces configured to directly contact a respective one of the pair of tissue holders to thereby position the outer skin surfaces of each edge of the incision or wound at locations relative to the opening at a distal end of the introducer.

The system further includes a controller operably coupled to the movable platform and the suture insertion device, the controller comprising a hardware processor coupled to non-transitory, computer-readable memory containing instructions executable by the processor to cause the controller to effect electro-mechanical control over: 1) movement of the platform to thereby cause corresponding movement of the suture insertion device relative to the incision or wound; 2) actuation of the first and second actuators to thereby cause movement of the pair of tissue holders; and 3) actuation of the insertion actuator to thereby cause deployment of one or more of the plurality of fasteners via the fastener delivery mechanism.

In some embodiments, the controller is programmed to coordinate movements of the pair of tissue holders until each tissue holder contacts a respective stopping surface with substantially the same predetermined force. The controller may further be programmed to sequentially actuate the insertion actuator to thereby actuate the fastener delivery mechanism to deliver one of the plurality of fasteners to a controlled distance relative to the opening at the distal end of the introducer and subsequently return first and second actuators and the insertion actuator to initial positions in preparation for deploying additional fasteners.

The controlled distance to which the fastener delivery mechanism moves relative to the distal end of the introducer can be determined via real-time analysis of a force applied by the insertion actuator.

In some embodiments, the predetermined force to which each tissue holder contacts a respective stopping surface is between about 8 Lbf and about 15 Lbf. In some embodiments, the predetermined force to which each tissue holder contacts a respective stopping surface provides a resistance to reverse movement of each tissue holder greater than about 20 Lbf/mm.

In some embodiments, the tracks for guiding the insertion device and the pair of tissue holders comprise a movable relationship with one another. For example, the pair of tissue holders may be configured to allow the tracks to move in response to forces applied upon the introducer such that the tracks are substantially centered between the pair of tissue holders.

In some embodiments, the opening in the introducer through which the bioabsorbable fastener passes is configured to adjust in size during and after deployment of a fastener. For example, the opening may be configured to change from a first size when a fastener is moving within the opening during deployment thereof, in which the sides of the opening directly contact the fastener, to a second different size after the fastener has been deployed, in which the opening is larger than the first size.

In some embodiments, that system may further include one or more tissue graspers operably coupled to the movable platform and configured to engage patient tissue and lift edges of the incision or wound to temporarily position each edge on opposite sides of the introducer of the suture insertion device in close proximity to the respective tissue holder on said given side of the introducer. The controller may be operably coupled to the one or more tissue graspers and configured to effect electro-mechanical control over the one or more tissue graspers. For example, the controller may be configured to control opening and closing of jaws of each respective tissue grasper and movement of the one or more tissue graspers.

In some embodiments, the system comprises three tissue graspers. For example, a first tissue grasper is configured to pinch tissue at an apex at a distal end of the incision or wound and the second and third tissue graspers are configured to pinch a respective opposing edge of the incision or wound closer to a proximal end of the incision or wound.

In some embodiments, the system includes a camera positioned to capture one or more images of an operating field, including capturing one or more images of tissue surrounding the incision or wound, the one or more tissue graspers, the edges of the incision or wound, and the introducer of the suture insertion device.

The controller is operably coupled to the camera and configured to receive image data therefrom and, based on analysis of said image data, the controller is configured to effect electro-mechanical control over at least one of: 1) movement of the one or more tissue graspers; 2) control over the opening and closing of a jaw of a given tissue grasper; 3) movement of the platform to thereby cause corresponding movement of the suture insertion device relative to the incision or wound; 4) actuation of the first and second actuators to thereby cause movement of the pair of tissue holders; and 5) actuation of the insertion actuator to thereby cause deployment of one or more of the plurality of fasteners via the fastener delivery mechanism.

### BRIEF DESCRIPTION OF THE DRAWINGS

In the drawings, like reference characters generally refer to the same parts throughout the different views. When components are symmetrical, the numbered features will be labeled with “a” for the left side and “b” for the right side, but in the text these symmetrical elements may be identified as “a,b”, even if only one component is visible in a particular drawing. Also, the drawings are not necessarily to scale, emphasis instead generally being placed upon illustrating the principles of the invention.

FIG. 1 shows a perspective view of an incision in skin.

FIG. 2 is close up of a cross section of one side of the incision of FIG. 1 taken along line 2-2 as it might appear being lifted by forceps according to the prior art.

FIG. 3 shows a cross section of an incision secured by a stitch according to the prior art.

FIG. 4 shows an embodiment of the present invention in proximity to a patient having an incision suitable for closure by surgical fasteners.

FIG. 5A is a perspective view of an embodiment of the present invention.

FIG. 5B is a perspective view similar to FIG. 5A showing the suture insertion device being removed/replaced.

FIGS. 6A, 6B, 6C, 6D illustrate various positions of an embodiment of the present invention in relation to the incision being closed.

FIG. 7 shows an embodiment of the suture insertion device portion of the present invention exploded to see the internal components.

FIG. 8 shows internal elements of the suture insertion device of an embodiment of the present invention as they might appear when separated from other parts of the system.

FIG. 9 shows the elements presented in FIG. 8, illustrated in the position assumed when ready to insert a fastener.

FIG. 10 is a graphical presentation of data from measurements of forces in various mechanism similar to and including an embodiment of the present invention.

FIG. 11 shows a cross section of one side of an incision as it might appear when positioned for fastener insertion by an embodiment of the present invention.

FIGS. 12A, 12B, 12C show three positions of an embodiment of the present invention to illustrate how the design of the delivery mechanism housings can reduce variability in the position of the fastener.

FIG. 13 is a graphical presentation of data from measurements of forces experienced when pushing a fastener into different tissues according to the present invention.

FIG. 14 illustrates the timing control chart for controlling the sequence of steps described in FIGS. 6A, 6B, 6C, 6D.

FIG. 15 shows a cross section of an incision secured by a bioabsorbable fastener according to the present invention.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present invention is directed to an automated skin closure system that operates with minimal input from skilled operators and without requiring a surgeon, thereby addressing the limitations of current methods and devices. The system is able to quickly deploy bioabsorbable fasteners to obtain a secure skin closure that will heal without complications and yield a minimal scar. The system makes it possible to automate skin closure for a large number of surgical procedures.

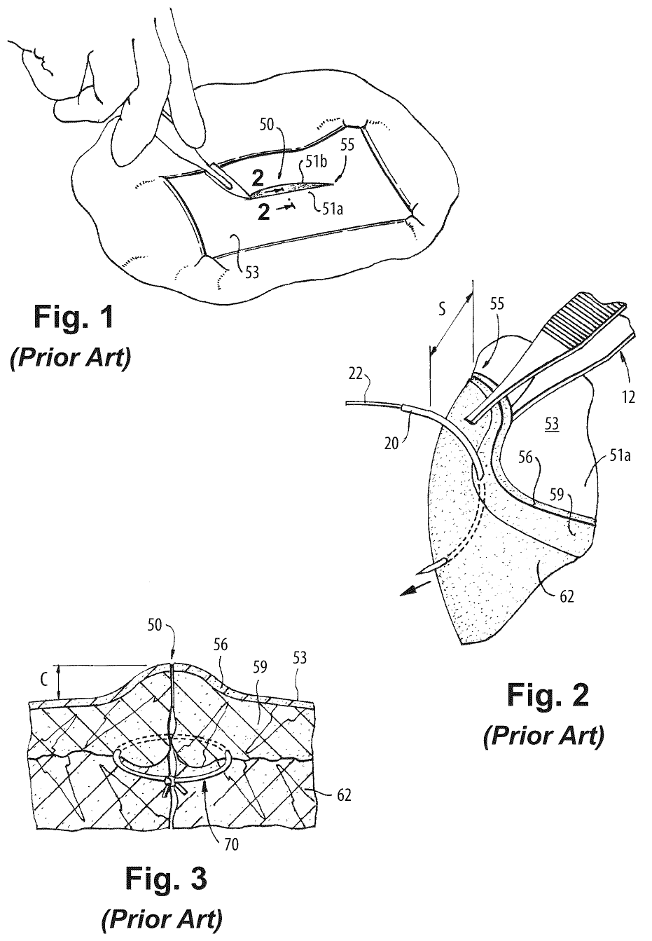

With reference to FIG. 1, an incision to be closed 50 is located in tissue having an outer skin surface 53. For purposes of this description, the “upward” or vertical direction is that direction generally perpendicular to the surface of the skin 53, even if that surface is curved or facing in another direction. Human skin is comprised of layers that can be seen in FIG. 2 which shows one side 51a of the incision of FIG. 1 in a cross-section taken along line 2-2. The outermost layer, the epidermis 56 consists of mostly dead cells. Below this is found the dermal layer 59 that is a thin layer of strong living tissue and then the subcutaneous layer 62.

FIG. 2 illustrates needle insertion according to the Set-back Buried Dermal Suture technique. Forceps 12 are used to lift one edge of the incision and a curved needle 20 connected to bioabsorbable suture material 22 is inserted a distance “S” back from the cut edge. The needle is held by a needle holder (not shown) and guided by the surgeon to enter and exit the dermal layer 59. The surgeon typically begins on the other side, 51b, passing the needle from deep to superficial, completing as shown in FIG. 2 moving from superficial to deep. This allows the cut ends of the suture material and the knot to remain deep in the incision, as shown in FIG. 3. The extent of the eversion “C” is determined by the distance that the stitch was set back from the cut edge and how small/tight the loop 70 is tied, variables that depend on the skill, experience, and judgement of the surgeon. Placing stitches and tying the knots with such painstaking precision is time consuming and has the risk of a needle stick accident.

As will be described in greater detail herein, the system of present invention automatically places a bioabsorbable suture beneath the skin surface, engaging the dermal layer on generally opposing sides of an incision to hold the incision closed with the optimal amount of eversion.

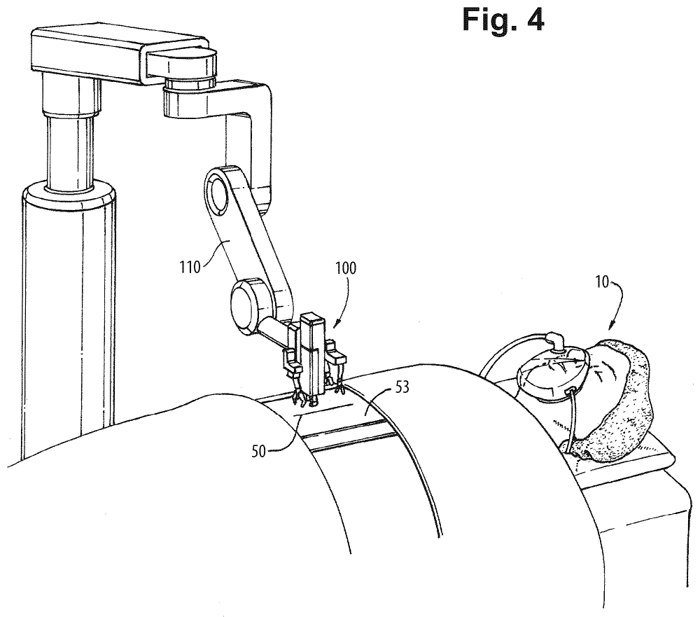

FIG. 4 shows an embodiment of an automated skin closure system 100 that is mounted on a robotic arm 110 similar to those mechanisms known to those skilled in the art. To make use of the system, the robotic arm is moved into a position that allows access to a patient 10 having an outer skin surface 53 with an incision 50 suitable for closure by surgical fasteners.

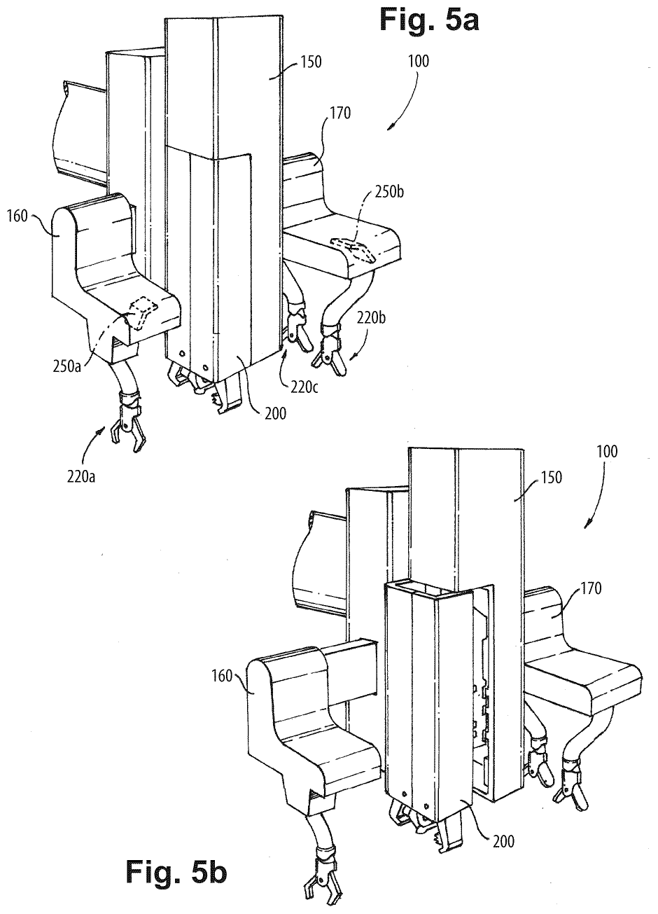

FIG. 5a shows one embodiment of the automated skin closure system 100 which comprises an actuator tower 150, a distal arm 160, and a proximal arm 170. The actuator tower 150 houses electro-motive actuators and is configured to receive a replaceable suture insertion device 200. The distal arm 160 supports a first grasper 220a and contains at least one vision system 250a. The proximal arm 170 supports second and third graspers 220b,c and houses an additional vision system 250b. The vision system(s) 250a, 250b may comprise one or more cameras, surface measuring lasers with associated detectors, and integrated circuit(s) and microprocessor(s) for carrying out instructions, including running analysis software, to thereby guide the operation of the automated skin closure system.

More specifically, the system 100 may include a programmable controller operably coupled to various components of the system 100, including the vision systems 250a, 250b, and configured to effect electro-mechanical control of various components based on inputs received via the vision systems 250a, 250b, and/or input from other sensors (e.g., force sensor(s), proximity sensor(s), etc.) associated with the system 100.

For example, the programmable controller may be operably coupled to at least one of: the actuator tower 150 and components thereof, the distal arm 160 and components thereof, the proximal arm 170 and components thereof, the first grasper 220a and components thereof, the second grasper 220b and components thereof, the third grasper 220c and components thereof, the suture insertion device 200 and components thereof, and each of the vision systems 250a, 250b. As described in greater detail herein, the controller may be configured to effect electro-mechanical control of one or more of the actuator tower, the graspers, and the suture insertion device, including actuation of one or more components of the suture insertion device, in response to input received via one or more sensors, including input from the vision system, to provide autonomous, or semi-autonomous, delivery of bioabsorbable fasteners from the suture insertion device to secure opposing sides of an incision and effectively close the incision and promote healing.

The programmable controller may generally include hardware processor(s) coupled to non-transitory, computer-readable memory containing instructions executable by the processor(s) to cause the controller to effect electro-mechanical control of the various components of the system 100. The controller includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more solid state drives), and the like. For example, memory can store computer-readable and computer-executable instructions or software for implementing/actuating components of the system 100, including the suture insertion device 200, or portions thereof. The controller also includes configurable and/or programmable processor(s) (e.g., central processing unit, graphical processing unit, etc.) and associated core(s) (e.g., in the case of computer systems having multiple processors/cores), for executing computer-readable and computer-executable instructions or software stored in the memory and other programs for controlling system hardware.

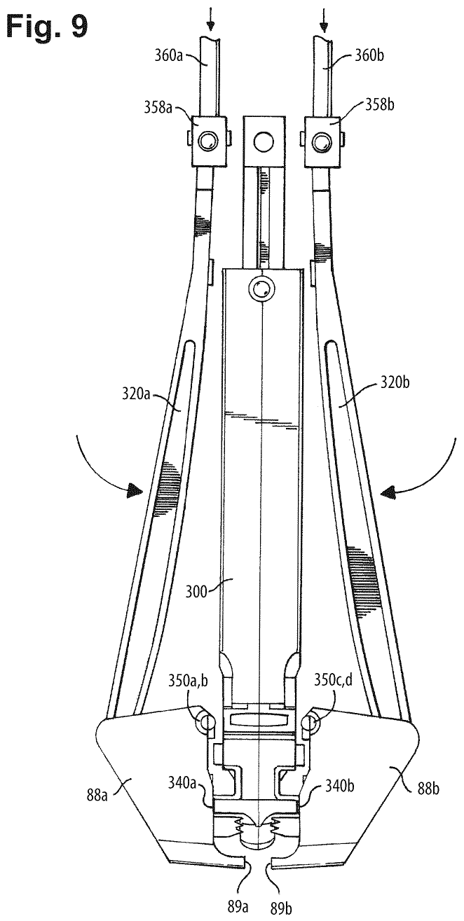

FIG. 5b shows the suture insertion device 200 separated from the actuator tower 150 as it might appear when being replaced. The suture insertion device 200 is a “one-time use” or “single-use” component and is designed to be replaced for each procedure. The distal arm 160 is extended to a “reloading” position to provide access for the user to replace the device 200. The operator loads the replacement of device 200 by guiding it into position so that tapered pins on the distal end of actuators within the actuator tower 150 are inserted into corresponding holes in the pushrods (shown in FIG. 9). Once inserted, proximity sensors (not shown) within the actuator tower 150 are configured to confirm proper positioning and the controller activates locking tabs to secure the suture insertion device 200 into position.

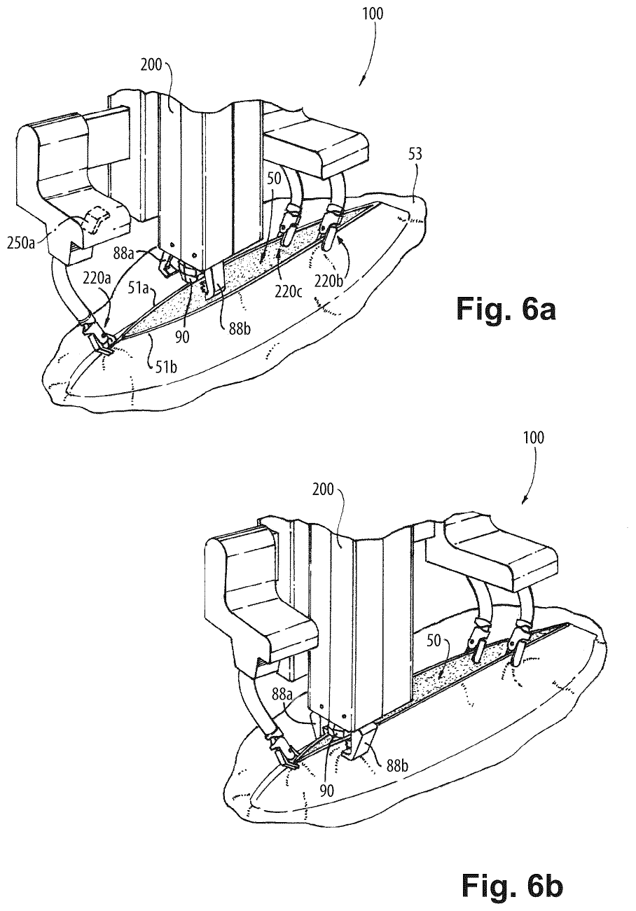

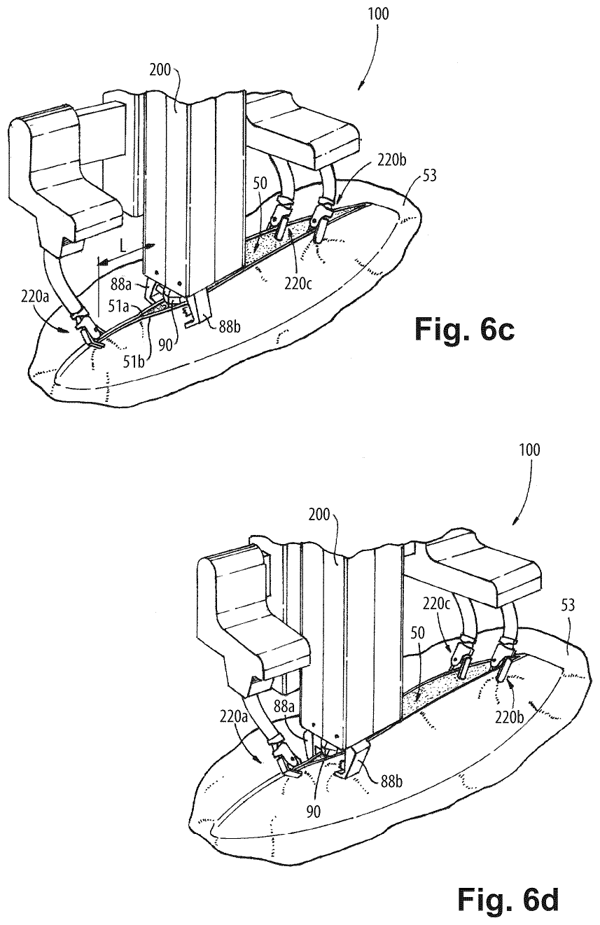

FIGS. 6a, 6b, 6c, and 6d illustrate the operational positions of the automated skin closure system 100 in relation to the outer skin surface 53 and the incision 50. For orientation, the left side of figure will be referred to as the distal end of the incision. Preferably, the incision is closed at the distal end first with subsequent insertions proceeding toward the right or proximal end.

As a first step, shown in FIG. 6a, the vision system 250a is activated to locate the incision. The boundaries of the incision may be identified by conventional pen-marks, adhesive dots or other digital identification means known in the art. Once the boundaries of the incision are confirmed, the controller aligns the automated skin closure system 100 with the longitudinal axis of the incision. The controller then directs the graspers to engage the tissue using feedback from the vision system. A first grasper 220a pinches the tissue at the apex at the distal end of the incision and second and third graspers 220b, 220c each pinch an edge of the incision approximately 50-60 mm from the apex. The graspers 220a,b,c lift the tissue a height above the plane of the outer skin 53. The distance that the edges are lifted is controlled by real-time analysis of feedback from graspers such that a maximum height of 10 mm or a lifting force to 5 lbs. (whichever is greater) is achieved at each grasper. With the tissue edges lifted and separated at the back to form a “V” shape, the introducer 90 is lowered between the tissue edges and moved to a position 5-10 mm from the distal apex.

FIG. 6b shows the automated skin closure system 100 as it would appear after completing a sequence of steps described in conjunction with FIG. 9. Tissue holders 88a,b are moved to their closed position and the suture insertion device 200 deploys a fastener, simultaneously capturing opposing sides of the incision.

FIG. 6c shows the automated skin closure system 100 after deploying a fastener and releasing the tissue holders 88a,b. The suture insertion device 200 is directed by the controller to move distally a predetermined distance “L” while maintaining the introducer 90 between the tissue edges 51a,b. The distance “L” is typically between 7 mm and 10 mm but can be greater or smaller based on user input and/or information specific to the procedure or patient. After moving the distance “L”, the tissue holders 88a,b momentarily pinch the tissue edges 51a,b to allow the graspers 220a,b,c to release and move.

FIG. 6d shows the automated skin closure system 100 as it would appear when deploying a second fastener. The graspers 220a,b,c have been moved the same distance “L” in concert with the movement of the suture insertion device 200, and are shown re-grasping the tissue edges 51a,b. This positions the first grasper 220a at the site where the first fastener was just deployed. Note that the relative positions of the graspers 220a,b,c and the suture insertion device 200 are the same as in FIG. 6b except that all are moved a distance “L” longitudinally along the axis of the incision. At this point the tissue holders 88a,b close and the fastener is inserted.

The steps 6b-6d of deploying a fastener, releasing, moving and re-grasping are repeated until the incision is closed. The graspers 220b,c that are farthest from the apex are disengaged and moved up/out of the field once the insertion point gets within 30 mm of the proximal end of the incision. For incisions shorter than 30 mm graspers 220b,c are used mainly to separate the sides of the incision to allow easy placement of the introducer 90 between the raised edges 51a,b.

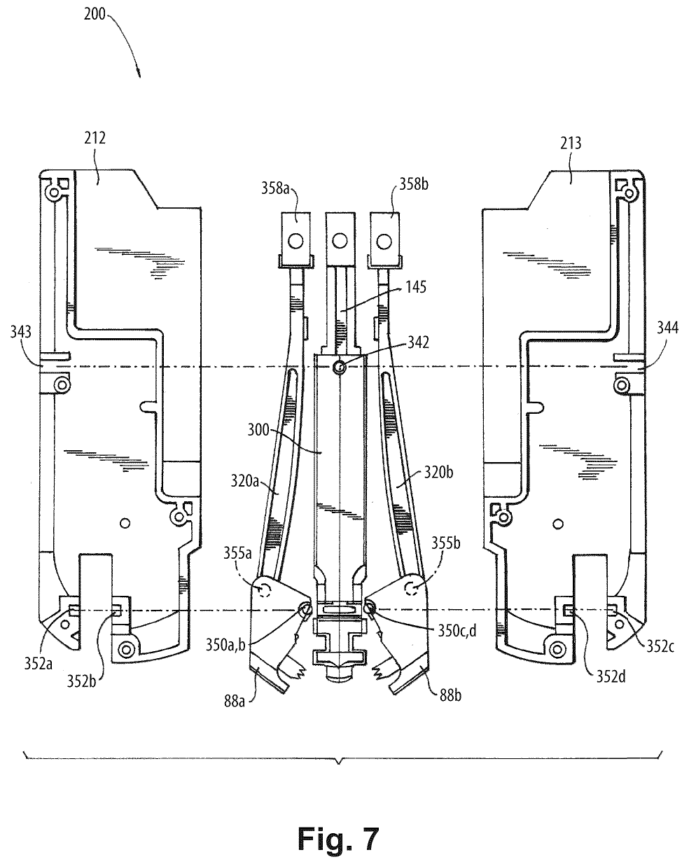

FIG. 7 shows an embodiment of the suture insertion device 200 exploded to see the internal components. The suture insertion device comprises a right shell 212, and a left shell 213 which can be assembled together to form a housing which orients and constrains the other elements. In a final assembly, the fastener delivery mechanism 300 resides within the assembled shells and is registered in position by balance pin 342 which is located in right receiver 343 and left receiver 344 when the shells are assembled. The fastener delivery mechanism 300 delivers fasteners one-by-one from a magazine containing a plurality of fasteners (not shown) each time the plunger needle assembly 145 is operated on by an actuator in the actuator tower. In some embodiments, the mechanical elements of the fastener delivery mechanism 300 are similar to those elements described in Rogers and essentially interact with the fasteners in a similar manner. The suture insertion device 200 also comprises tissue holders 88a,b which are principally registered in the shells by tissue holder pivot pins 350a,b,c,d which are located in right foot receivers 352a,b and left foot receivers 352c,d for the right and left tissue holders respectively. Each tissue holder 88a,b is rotatably coupled to the distal end of a pushrod 320a,b which applies force to the tissue holder through contact with tissue holder push-bar 355a,b. Connectors 358a,b at the proximal ends of the pushrods 320a,b are configured to interface with actuators in the actuator tower 150 (not shown).

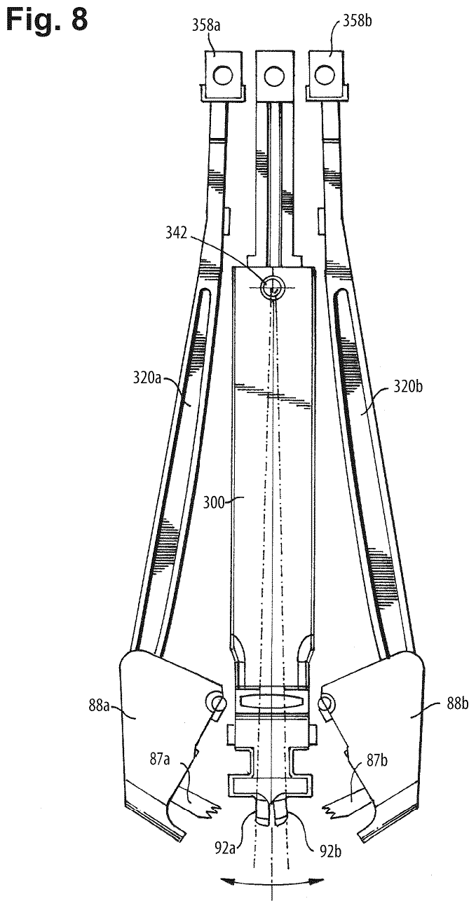

FIG. 8 shows internal elements of the suture insertion device as they might appear when separated from the right and left shells. Force from actuators (not shown) is applied to connectors 358a,b at the proximal ends of the pushrods 320a,b to cause rotation of the tissue holders 88a,b. The tissue holders comprise pincers 87a,b which press against the outer surface of the tissue edges to pinch the tissue against pinch rails 92a,b. As the tissue holders move to position the tissue for fastener insertion, it is critical that the tissue edges are presented equally on both left and right sides of the device to obtain reliable bilateral capture of the dermal layers. Even small differences in dimensions left to right can cause one leg of the fastener to miss the dermal layer. This can happen, for example, if one side is undermined differently or if scar tissue was removed from one side and not the other. One embodiment of the present invention addresses such imbalances by providing a centering mechanism that ensures equal spacing between the fastener and the foot on each side of the incision. In this embodiment the fastener delivery mechanism 300 is rotatably attached at its proximal end to the shells of the suture insertion device 200 using a cylindrical balance pin 342. When the pincers 87a,b press tissue against the pinch rails 92a,b any initial imbalance in the force being applied by one side versus the other is neutralized by the movement of the delivery mechanism until the forces are balanced. In this way the fastener delivery mechanism and the tracks guiding the insertion device are centered between the tissue holders/feet.

FIG. 9 shows the same elements of the suture insertion device as previously presented in FIG. 8 but now illustrated in the position assumed when ready to insert the fastener. The tissue holders 88a,b are connected to pushrods 320a,b which are operatively coupled to actuators 360a,b via pushrod connectors 358a,b. The actuators 360a,b provide a driving force aligned with the longitudinal axis of the pushrods to cause the tissue holders to rotate about tissue holder pivot pins 350a,b,c,d and to move until they contact stops 340a,b located at the distal end of the fastener delivery mechanism 300. Contact with the stops 340a,b blocks further movement of the tissue holders 88a,b so that the frontal surfaces of the feet 89a,b where the feet contact the outer skin surface, are positioned to create a gap of predetermined size. To ensure that the size of the gap is maintained during insertion of the fastener, the actuators 360a,b continue to move a small amount after the tissue holders 88a,b contact the stops 340a,b. The amount of movement is controlled using force and position sensors on the actuators as feedback. Force is applied equally to right and left pushrods 320a,b to reach a predetermined force and/or distance that provides sufficient stiffness to resist reverse rotation of the tissue holders 88a,b. We have determined that a stiffness, the force gradient that resists movement of the frontal surface of the tissue holder/foot 89a,b, of more than 20 Lbf/mm is needed to achieve reliable positioning of the fastener in the dermal tissue. In one embodiment the required stiffness is obtained by applying sufficient force to bend the pushrods as indicated by the curved arrows in FIG. 9. The pushrods 320a,b are fabricated from a strong, resilient material such as 30% glass filled nylon so that when they are bent they have a restorative force which is able to resist reverse rotation of the tissue holders 88a,b.

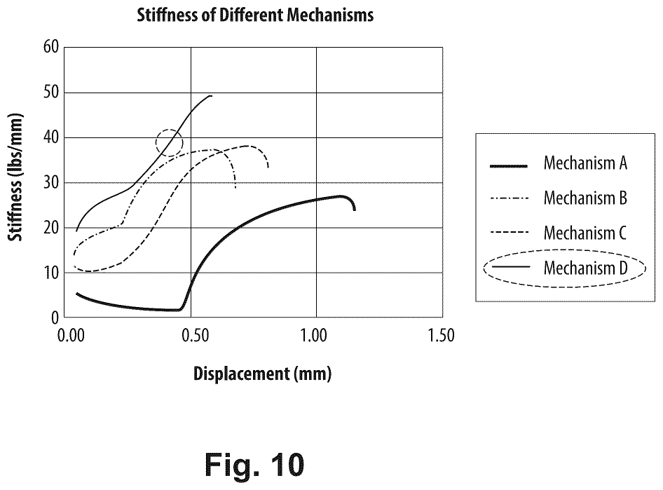

Alternative mechanical linkages may be created to move the tissue holders by those skilled in the art. The alternate linkages can be tested by measuring the force needed to separate the frontal surfaces of the feet 89a,b to determine if they have the stiffness required of the present invention. Stiffness is the derivative of the force-displacement curve. FIG. 10 illustrates the results of measuring the force needed to increase the gap between frontal surfaces of the feet 89a,b when the mechanism is positioned for deployment of a fastener. Mechanism D represents the present invention and achieves a stiffness of almost 40 Lbf/mm at a displacement of 0.4 mm (circle area of chart).

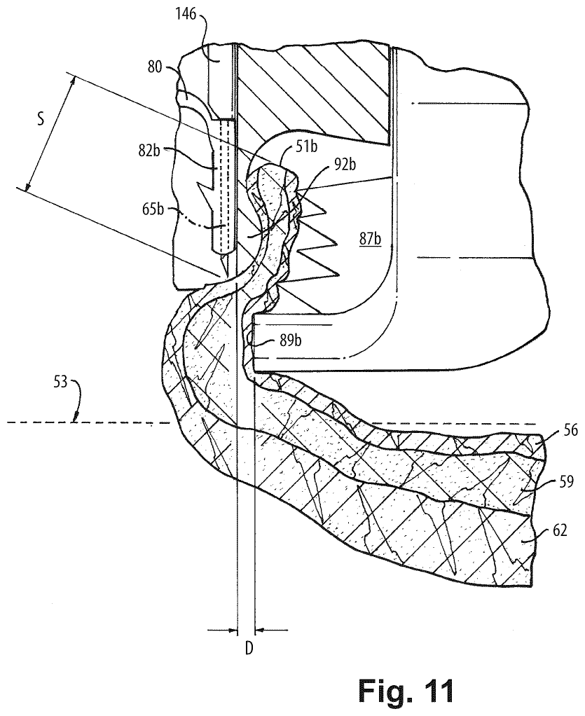

FIG. 11 shows a cross section of one side of an incision 51b positioned for fastener insertion. The edge of the incision is secured by being pinched between pincer 87b and pinch rail 92b. One leg 82b of the fastener 80 is shown mounted on needle 65b and carried or pushed by insertion device 146. An actuator (not shown) within the actuator tower is operatively coupled to move insertion device 146 along an axis substantially perpendicular to the skin surface 53. To deploy a fastener, the actuator advances distally to move all components together: insertion device 146, fastener 80, and needle 65b. The movement causes the tip of the needle 65b to penetrate subcutaneous tissue 62 and the dermal layer 59 of the tissue. The figure shows “S” which is the distance between the point where the fastener pierces the tissue and the cut edge 51b. “S” has the same meaning as in the Set-Back Buried Dermal Suture technique shown in FIG. 2. The distal surface of the foot 89b of the tissue holder presses against the outer skin surface and moves to a predetermined position relative to the path of the fastener. We have determined that “D”, the distance between the outer-most surface of the fastener leg 82b and the distal surface of the foot where it contacts the outer skin surface 89b, should not exceed 0.27 mm and should typically be 0.17 mm. The dermal layer, which because of its strength is the target for inserting the fastener, is immediately below the epidermis 56. Since the epidermis is thin and in direct contact with the foot portion of the tissue holder, variation in the epidermis will displace the dermal tissue only a tiny fraction of a millimeter. Therefore, by positioning the outer skin surface a distance “D” from the fastener's outer-most surfaces, where “D” is between 0.17 mm and 0.27 mm, the fastener will engage dermis independent of the thickness of the skin tissue and its various layers.

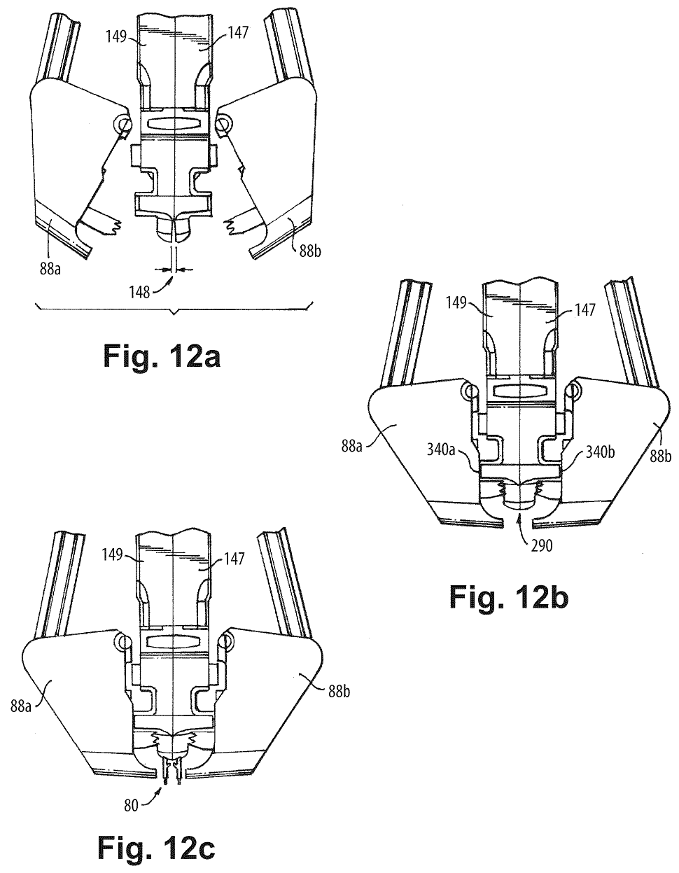

The discussion with reference to FIG. 11 emphasized the importance of accurately directing the point at which the fastener pierces the tissue. The inventive elements of the present invention described in reference to FIG. 8 and FIG. 9 ensure precise placement of the tissue, but the fastener 80 also must be placed with similar precision. FIGS. 12a-12c show three positions of an embodiment of the present invention to illustrate how the design of the delivery mechanism can reduce variability in the position of the fastener. The components of the fastener delivery mechanism 300 are contained within two parts: the delivery mechanism left housing 147 and the delivery mechanism right housing 149. When the two housings are assembled, guide-tracks are formed in which the plunger needle assembly and the insertion device 146 (not shown), slide to deliver fasteners one at a time. By conventional designs, the tracks would be wider than the width of the insertion device carrying the fastener, to facilitate sliding. This sliding space (typically +/−0.1 mm) may also include tolerance for part variation (typically +/−0.06 mm). The end result can allow significant variation in the path of the fastener when compared to the very small target that it must hit. The present invention reduces that variation by making guide-tracks that are NOT wider than the insertion device and fastener; in fact, making them slightly narrower. To avoid high friction, delivery mechanism left housing 147 and delivery mechanism right housing 149 are assembled with a small gap 148 between them at the distal end as shown in FIG. 12a. At the beginning of an insertion, the insertion device with the fastener slides easily in the guide-tracks because the gap 148 provides clearance for the movement. As the fastener continues movement and reaches the exit 290, the tissue holders 88a,b make contact with the stops 340a,b and force the gap 148 to be reduced to zero (see FIG. 12b). This means that the fastener is guided by line-to-line contact which minimizes positional uncertainty as it traverses the exit and penetrates the tissue. The added frictional force encountered in the final few millimeters of travel during insertion is small compared to the force required to penetrate the tissue. Therefore, actuator force is able to deliver the fastener into the tissue with line-to-line guiding precision (see FIG. 12c). To complete the insertion cycle, the insertion actuator, controlling the insertion device, retracts a short distance to remove the needles from the tissue. The actuators controlling the pushrods 320a,b are then reversed which immediately releases the force applied by the tissue holders 88a,b on the stops 340a,b. The release of a clamping force on the stops 340a,b allows the gap 148 to open, thereby returning the device to the configuration shown in FIG. 12a. With the gap re-established, the insertion device can be easily withdrawn by the insertion actuator in preparation for deploying the next fastener.

Deployment of the fastener into the tissue has another dimension that must be controlled to achieve reliable automated skin closure. That dimension is the distance that the fastener is pushed before the actuator reverses and withdraws the insertion device. Fasteners of the type taught by Danielson have a frontal surface area considerably larger than the needle. That surface area is increased further if the fastener has barbs as in one embodiment of the present invention. When the needle and fastener enter the tissue, the force required to penetrate increases with each increase in frontal surface area. This causes the tissue to stretch away from the insertion device thus requiring the actuator to push further. At some distance, which varies according to the characteristics of the tissue, the fastener/barb penetrates the dermis. If the actuator which delivers the fastener stops short of this distance, it may result in poor engagement of the fastener and an unsatisfactory closure.

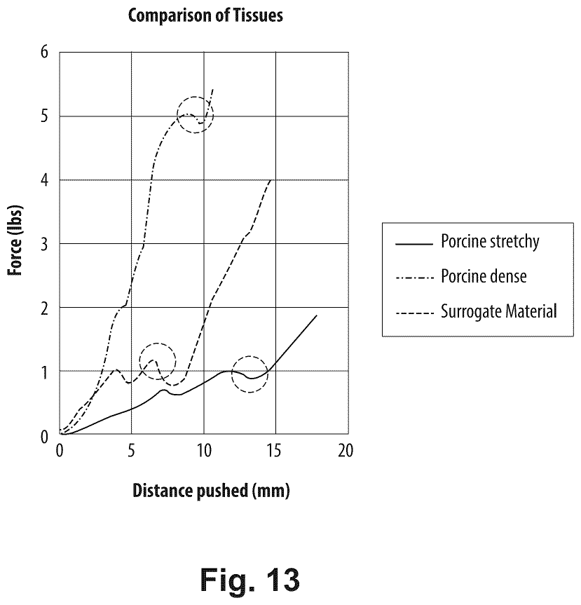

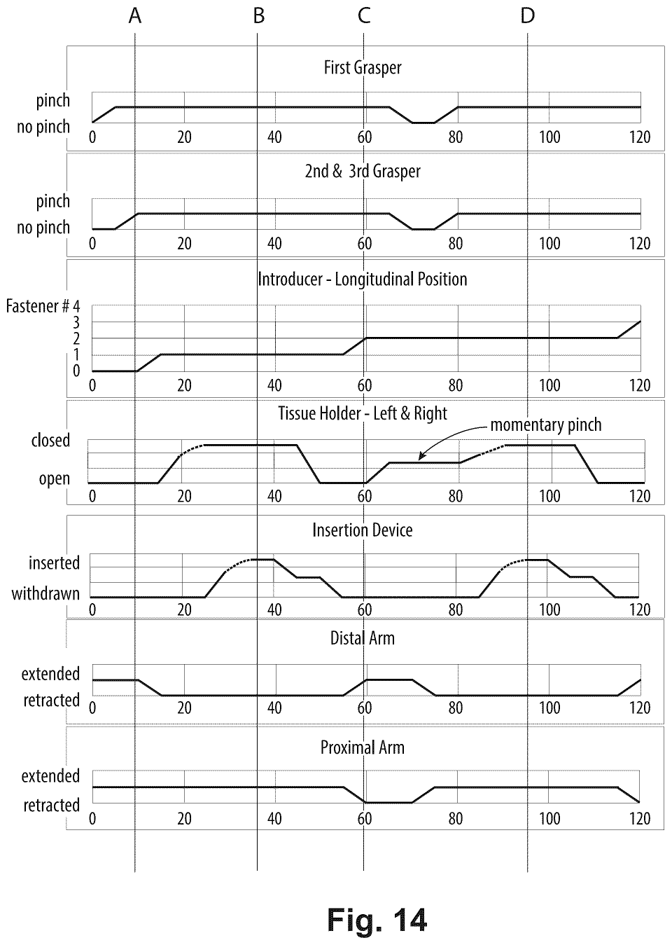

One embodiment of the present invention adjusts the distance that the fastener is pushed according to the properties of the tissue. FIG. 13 shows the forces exerted by the actuators at various distances as a fastener is pushed into tissues with varying properties. Note the two ‘bumps’ on the curve for Surrogate Material at distances of approximately 4 mm and 6 mm as shown in the chart of FIG. 13. The first occurs when the tissue expands from the needle entry to accommodate the front tip of the fastener. The second bump (circled) occurs when the barb enters. The ‘bump’ is created due to the elastic properties of skin tissue, which is initially stretched and then rebounds once the fastener penetrates. Note that the distances for penetration, indicated by dotted circles for each tissue material, are significantly greater for the Porcine stretchy tissue. Conversely the distance to penetrate Porcine dense tissue, as shown in this experiment, is shorter but requires significantly more force. The present invention uses real-time analysis of data similar to that shown in FIG. 13 to control the distance that the fastener is pushed. A change in slope of the force curve in the region where penetration is expected, is used as a signal to halt and reverse the actuator. Thus, in the case of the experimental data shown in FIG. 13, the actuator would drive the insertion device to distances of 13 mm, 9.5 mm and 7.5 mm for the stretchy, dense, and surrogate tissues respectively. Additional refinements of control may be implemented by those skilled in the art to include, limiting distance for safety, detecting errors, and providing expected distances based on the surgical site and patient demographic information which can be provided to the automated system.

FIG. 14 illustrates the timing control chart for controlling the sequence of steps described in FIG. 6a-6d. The x-axis is marked with arbitrary timing intervals. The initial steps which were illustrated in FIGS. 6a and 6b are illustrated in the interval 0-55. The interval 60-115 illustrates the steps as shown in FIGS. 6c and 6d. The Chart indicates the exact time captured in FIGS. 6a, 6b, 6c, 6d with the labels A, B, C, D shown at the top of FIG. 14. The dotted segments of the line labeled Tissue Holder-Left & Right at intervals 20-25 and 85-90 indicate the additional movement of the tissue holders which is variable according to feedback from force and position sensors as described in conjunction with FIG. 9. The dotted segments in the line labeled Insertion Device at 30-35 and 90-95 indicate the additional distance that the fastener is pushed which is variable according to feedback as described in conjunction with FIG. 13. The line labeled Introducer-Longitudinal Position indicates the sequential fastener insertions, beginning at the apex of the incision and moving one-by-one as the fasteners are deployed. After each insertion when the insertion device is withdrawn (see Insertion Device line at 55 for example), the distal and proximal arms move in concert to reposition the actuator tower 150 thus moving the introducer to the next fastener insertion position (at time interval 60). At timing interval 65, the chart illustrates the movement of the tissue holders to apply a partial/momentary pinch so that first grasper and 2^{nd} and 3^{rd} graspers can reposition. Once repositioned, the tissue holders are fully closed, followed by the insertion device inserting the next fastener (time interval 85-100). The system reaches a stopping point after each insertion (for example at timing interval 115). If no more fasteners are needed, the actuator tower 150 is lifted and the system is removed. If additional fasteners are needed the system repeats restarting at 55.

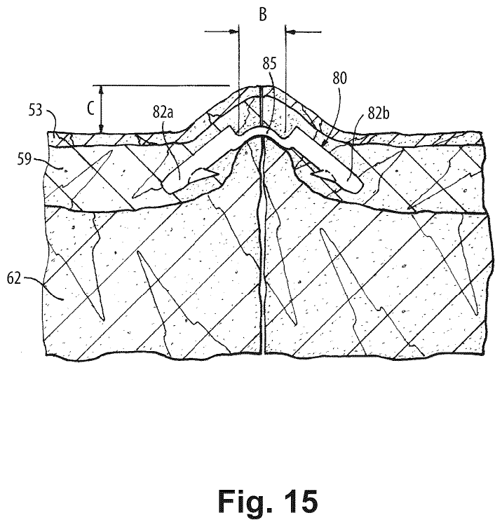

FIG. 15 illustrates in cross section, a fastener after insertion by an embodiment of the present invention. A bioabsorbable fastener 80, having two legs 82a,b joined by a flexible connecting bridge 85, engages the dermal tissue 59, to hold the edges of the incision closed in much the same manner as the set-back stitch shown in FIG. 3. The dimension “B” is the length of the connecting bridge 85, which plays a critical role in determining the amount of eversion “C”. The amount of eversion “C” is determined by the formula C=S−B/2 where S is the set-back distance shown in FIG. 11 and B is the length of the connecting bridge shown in FIG. 15., with all variables expressed in millimeters. The dimension “S” may vary slightly by the amount of tissue that is captured in the pinching process, but this is monitored by the vision system after each fastener insertion and may be adjusted to minimize variation. By minimizing variation in “S” and employing a fastener with known dimension “B”, a uniform amount of eversion “C” is achieved.

As used in any embodiment herein, the term “controller”, “computing system”, “computing device”, and “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An automated system for closing one or more incisions or wounds of a patient, the system comprising:
a movable platform;
a suture insertion device releasably coupled to a portion of the movable platform, the suture insertion device configured to deploy bioabsorbable fasteners to close an incision or wound in tissue of a patient, the suture insertion device comprising:
a body comprising a pair of tissue holders positioned adjacent to a distal end of the body and operably coupled to first and second actuators associated with the movable platform, each of the first and second actuators being configured to move the respective tissue holder to effectively pinch and fold one edge of the incision or wound in preparation for deployment of a fastener; and
a fastener delivery mechanism operably coupled to an insertion actuator associated with the movable platform and configured to deploy one or more of a plurality of bioabsorbable fasteners, wherein the fastener delivery mechanism comprises:
an insertion device comprising one or more insertion needles, said insertion device being guided within tracks and configured to move in response to movement of the insertion actuator to cause the one or more insertion needles to releasably engage and deploy each one of the plurality of fasteners in a one-by-one fashion in response to repeated movements of the insertion actuator;
an introducer positioned at, and extending from, a distal end of the fastener delivery mechanism, the introducer configured to directly contact opposing edges of the wound or incision and having an opening through which a fastener exits the distal end of the introducer during deployment; and
a pair of stopping surfaces configured to directly contact a respective one of the pair of tissue holders to thereby position the outer skin surfaces of each edge of the incision or wound at locations relative to the opening at a distal end of the introducer; and
a controller operably coupled to the movable platform and the suture insertion device, the controller comprising a hardware processor coupled to non-transitory, computer-readable memory containing instructions executable by the processor to cause the controller to effect electro-mechanical control over:
movement of the platform to thereby cause corresponding movement of the suture insertion device relative to the incision or wound;
actuation of the first and second actuators to thereby cause movement of the pair of tissue holders; and
actuation of the insertion actuator to thereby cause deployment of one or more of the plurality of fasteners via the fastener delivery mechanism.

2. The system of claim 1, wherein the controller is programmed to coordinate movements of the pair of tissue holders until each tissue holder contacts a respective stopping surface with substantially the same predetermined force.

3. The system of claim 2, wherein the controller is further programmed to sequentially actuate the insertion actuator to thereby actuate the fastener delivery mechanism to deliver one of the plurality of fasteners to a controlled distance relative to the opening at the distal end of the introducer and subsequently return first and second actuators and the insertion actuator to initial positions in preparation for deploying additional fasteners.

4. The system of claim 3, wherein the controlled distance to which the fastener delivery mechanism moves relative to the distal end of the introducer is determined via real-time analysis of a force applied by the insertion actuator.

5. The system of claim 2, wherein the predetermined force to which each tissue holder contacts a respective stopping surface is between about 8 Lbf and about 15 Lbf.

6. The system of claim 2, wherein the predetermined force to which each tissue holder contacts a respective stopping surface provides sufficient resistance to reverse movement of each tissue holder.

7. The system of claim 6, wherein the resistance to reverse movement is greater than about 20 Lbf/mm.

8. The system of claim 1, wherein the tracks for guiding the insertion device and the pair of tissue holders comprise a movable relationship with one another.

9. The system of claim 8, wherein the pair of tissue holders are configured to allow the tracks to move in response to forces applied upon the introducer such that the tracks are substantially centered between the pair tissue holders.

10. The system of claim 1, wherein the opening in the introducer through which the bioabsorbable fastener passes is configured to adjust in size during and after deployment of a fastener.

11. The system of claim 10, wherein the opening is configured to change from a first size when a fastener is moving within the opening during deployment thereof, in which the sides of the opening directly contact the fastener, to a second different size after the fastener has been deployed, in which the opening is larger than the first size.

12. The system of claim 1, further comprising one or more tissue graspers operably coupled to the movable platform and configured to engage patient tissue and lift edges of the incision or wound to temporarily position each edge on opposite sides of the introducer of the suture insertion device in close proximity to the respective tissue holder on said given side of the introducer.

13. The system of claim 12, wherein the controller is operably coupled to the one or more tissue graspers and configured to effect electro-mechanical control over the one or more tissue graspers.

14. The system of claim 13, wherein the controller is configured to control opening and closing of jaws of each respective tissue grasper and movement of the one or more tissue graspers.

15. The system of claim 13, wherein the system comprises three tissue graspers.

16. The system of claim 14, wherein a first tissue grasper is configured to pinch tissue at an apex at a distal end of the incision or wound and the second and third tissue graspers are configured to pinch a respective opposing edge of the incision or wound closer to a proximal end of the incision or wound.

17. The system of claim 13, further comprising a camera positioned to capture one or more images of an operating field, including capturing one or more images of tissue surrounding the incision or wound, the one or more tissue graspers, the edges of the incision or wound, and the introducer of the suture insertion device.

18. The system of claim 17, wherein the controller is operably coupled to the camera and configured to receive image data therefrom and, based on analysis of said image data, the controller is configured to effect electro-mechanical control over at least one of:
movement of the one or more tissue graspers;
control over the opening and closing of a jaw of a given tissue grasper;
movement of the platform to thereby cause corresponding movement of the suture insertion device relative to the incision or wound;
actuation of the first and second actuators to thereby cause movement of the pair of tissue holders; and
actuation of the insertion actuator to thereby cause deployment of one or more of the plurality of fasteners via the fastener delivery mechanism.

19. The system of claim 1, wherein the suture insertion device is a single use device.

20. The system of claim 19, wherein the movable platform is configured to receive and releasably retain interchangeable suture insertion devices.
